(19) United States
(12) Patent Application Publication
(10) Pub. No.: US 2004/0186021 A1
(43) Pub. Date: Sep. 23, 2004

(54) Substituted benzo-nitro-heterocycles

(76) Inventors: Otto Schallner, Monheim (DE); Dorothee Hoischen, Düsseldorf (DE); Mark Wilhelm Drewes, Langenfeld (DE); Peter Dahmen, Neuss (DE); Dieter Feucht, Monheim (DE); Rolf Pontzen, Leichlingen (DE)

Correspondence Address: BAYER CROPSCIENCE LP, Patent Department, 100 BAYER ROAD, PITTSBURGH, PA, 15205-9741, US

(21) Appl. No.: 10/483,359

(22) PCT Filed: Jul. 12, 2001
(86) PCT No.: PCT/EP02/07230

(30) Foreign Application Priority Data
Jul. 12, 2001 (DE) 101 33 691.8

(51) Int. Cl. A01N 43/72; A01N 43/54; C07D 417/02; C07D 413/02; C 07D 4 3/02

(52) U.S. Cl. 504/221; 504/223; 504/239; 544/105; 544/295; 544/50

(57) ABSTRACT

The invention relates to novel substituted benzo-nitrogen heterocycles of the formula (I)

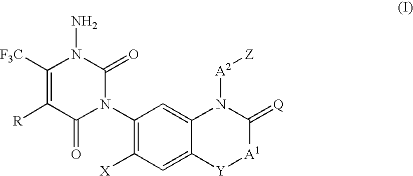

in which

A¹, A², Q, R, X, Y and Z are as defined in the description,

and to a process for their preparation and to their use as plant treatment agents.

The invention relates to novel substituted benzo-nitrogen heterocycles, to a process for their preparation, and to their use as plant treatment agents, in particular as herbicides.

A large number of benzo-nitrogen heterocycles having herbicidal properties have already been disclosed (cf. EP-A-408382, EP-A-420194, EP-A-489480, U.S. Pat. No. 4,981,508, U.S. Pat. No. 5,084,084, U.S. Pat. No. 5,127,935, U.S. Pat. No. 5,314,864, WO-A-90/15057). However, the action of these compounds is not satisfactory in all respects.

This invention now provides the novel substituted benzo-nitrogen heterocycles of the formula (I)

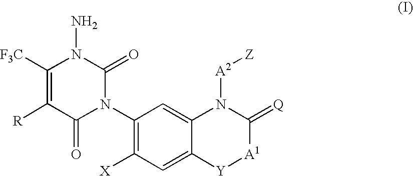

in which

A¹ represents a single bond or represents straight-chain or branched alkane diyl having 1 to 4 carbon atoms,

A² represents a single bond or represents straight-chain or branched alkanediyl having 1 to 4 carbon atoms or represents optionally cyano-, carboxyl-, halogen-, C₁-C₄-alkoxy or C₁-C₄-alkoxy-carbonyl-substituted alkene diyl having 2 to 6 carbon atoms,

Q represents O (oxygen) or S (sulphur),

R represents hydrogen, cyano, halogen or optionally halogen-substituted straight-chain or branched alkyl having 1 to 4 carbon atoms,

X represents hydrogen or halogen,

Y represents O (oxygen), S (sulphur), NH or N-(C₁-C₄-alkyl), and

Z represents cyano, one of the groupings —CQ¹-O—R¹, —CQ²-N(R²,R³), —C(R⁴)═O, —C(R⁴)═N—O—R⁵, or represents alkylsulphonyl, alkoxysuphonyl or dialkylaminosulphonyl having in each case 1 to 6 carbon atoms in the alkyl groups, where

Q¹ represents O (oxygen) or S (sulphur),

Q² represents O (oxygen) or S (sulphur),

R¹ represents hydrogen, optionally cyano-, carboxyl-, halogen-, C₁-C₄ alkoxy- or C₁-C₄-alkoxy-carbonyl-substituted straight-chain or branched alkyl having 1 to 10 carbon atoms, in each case optionally cyano-, carboxyl-, halogen- or C₁-C₄-alkoxy-carbonyl-substituted straight-chain or branched alkenyl or alkynyl having in each case 3 to 10 carbon atoms, in each case optionally cyano-, carboxyl-, halogen- or C₁-C₄-alkoxy-carbonyl-substituted cycloalkyl or cycloalkylalkyl having in each case 3 to 6 carbon atoms in the cycloalkyl group and optionally 1 to 4 carbon atoms in the alkyl moiety, or represents in each case optionally nitro-, cyano-, carboxyl-, carbamoyl-, C₁-C₄-alkyl-, C₁-C₄-halogenoalkyl-, C₁-C₄-alkoxy-, C₁-C₄-halogenoalkoxy- or C₁-C₄-alkoxy-carbonyl-substituted aryl or arylalkyl having in each case 6 or 10 carbon atoms in the aryl group and optionally 1 to 4 carbon atoms in the alkyl moiety,

R² represents hydrogen, optionally cyano-, carboxyl-, halogen-, C₁-C₄-alkoxy- or C₁-C₄-alkoxy-carbonyl-substituted straight-chain or branched alkyl having 1 to 10 carbon atoms, in each case optionally cyano-, carboxyl-, halogen- or C₁-C₄-alkoxy-carbonyl-substituted straight-chain or branched alkenyl or alkynyl having in each case 3 to 10 carbon atoms, or represents in each case optionally cyano-, carboxyl-, halogen- or C₁-C₄-alkoxy-carbonyl-substituted cycloalkyl or cycloalkylalkyl having in each case 3 to 6 carbon atoms in the cycloalkyl group and optionally 1 to 4 carbon atoms in the alkyl moiety,

R³ represents hydrogen, optionally cyano-, carboxyl, halogen-, C₁-C₄-alkoxy- or C₁-C₄-alkoxy-carbonyl-substituted straight-chain or branched alkyl having 1 to 10 carbon atoms, in each case optionally cyano-, carboxyl-, halogen- or C₁-C₄-alkoxy-carbonyl-substituted straight-chain or branched alkenyl or alkynyl having in each case 3 to 10 carbon atoms, in each case optionally cyano-, carboxyl-, halogen- or C₁-C₄-alkoxy-carbonyl-substituted cycloalkyl or cycloalkylalkyl having in each case 3 to 6 carbon atoms in the cycloalkyl group and optionally 1 to 4 carbon atoms in the alkyl moiety, or represents in each case optionally nitro-, cyano-, carboxyl-, carbamoyl-, C₁-C₄-alkyl-, C₁-C₄-halogenoalkyl-, C₁-C₄-alkoxy-, C₁-C₄-halogenoalkoxy- or C₁-C₄-alkoxy-carbonyl-substituted aryl or arylalkyl having in each case 6 or 10 carbon atoms in the aryl group and optionally 1 to 4 carbon atoms in the alkyl moiety,

R⁴ represents hydrogen or represents optionally cyano-, halogen- or C₁-C₄-alkoxy-substituted alkyl having 1 to 6 carbon atoms, and

R⁵ represents hydrogen or alkyl having 1 to 6 carbon atoms.

Saturated or unsaturated hydrocarbon radicals, such as alkyl, alkenyl, alkynyl, alkanediyl or alkenediyl, are in each case straight-chain or branched as far as this is possible—including in combination with heteroatoms, such as in alkoxy.

Optionally substituted radicals can be mono- or polysubstituted, where in the case of polysubstitution, the substituents can be identical or different.

If appropriate, the compounds of the general formula (I) according to the invention contain one or more asymmetrically substituted carbon atoms, in which case they can be present in different enantiomeric (R- and S-configured) forms or diastereomeric forms. In these cases, the invention relates both to the different possible individual enantiomeric or stereoisomeric forms of the compounds of the general formula (I) and to the mixtures of these isomeric compounds.

Preferrred substituents or preferred ranges of the radicals present in the formulae given above and below are defined below.

A¹ preferably represents a single bond or represents straight-chain or branched alkanediyl having 1 to 3 carbon atoms.

A² preferably represents a single bond or represents straight-chain or branched alkanediyl having 1 to 3 carbon atoms or represents optionally cyano-, carboxyl-, fluorine-, chlorine-, bromine-, methoxy-, ethoxy-, n- or i-propoxy-, n-, i-, s- or t-butoxy-, methoxycarbonyl-, ethoxycarbonyl-, n- or i-propoxycarbonyl-, n-, i-, s- or t-butoxycarbonyl-substituted alkenediyl having 2 to 4 carbon atoms.

Q preferably represents O (oxygen).

R preferably represents hydrogen, cyano, halogen or optionally halogen-substituted straight-chain or branched alkyl having 1 to 3 carbon atoms.

X preferably represents hydrogen, fluorine, chlorine or bromine.

Y preferably represents O (oxygen), S (sulphur) or NH.

Z preferably represents cyano, one of the groupings —CQ¹-O—R¹, —CQ²-N(R²,R³), —C(R⁴)═O, —C(R⁴)═N—O—R⁵, or represents alkylsulphonyl, alkoxysulphonyl or dialkylaminosulphonyl having in each case 1 to 4 carbon atoms in the alkyl groups.

Q¹ preferably represents O (oxygen).

Q² preferably represents O (oxygen).

R¹ preferably represents hydrogen, optionally cyano-, carboxyl-, halogen, C₁-C₃-alkoxy- or C₁-C₃-alkoxy-carbonyl-substituted straight-chain or branched alkyl having 1 to 6 carbon atoms, in each case optionally cyano-, carboxyl-, halogen- or C₁-C₃-alkoxy-carbonyl-substituted straight-chain or branched alkenyl or alkynyl having in each case 3 to 6 carbon atoms, in each case optionally cyano-, carboxyl-, halogen- or C₁-C₃-alkoxy-carbonyl-substituted cycloalkyl or cycloalkylalkyl having in each case 3 to 6 carbon atoms in the cycloalkyl group and optionally 1 to 3 carbon atoms in the alkyl moiety, or represents in each case optionally nitro-, cyano-, carboxyl-, carbamoyl-, C₁-C₄-alkyl-, C₁-C₃-halogenoalkyl-, C₁-C₃-alkoxy-, C₁-C₃-halogenoalkoxy- or C₁-C₃-alkoxy-carbonyl-substituted aryl or arylalkyl having in each case 6 or 10 carbon atoms in the aryl group and optionally 1 to 3 carbon atoms in the alkyl moiety.

R² preferably represents hydrogen, optionally cyano-, carboxyl-, halogen-, C₁-C₃-alkoxy- or C₁-C₃-alkoxy-carbonyl-substituted straight-chain or branched alkyl having 1 to 6 carbon atoms, in each case optionally cyano-, carboxyl-, halogen- or C₁-C₃-alkoxy-carbonyl-substituted straight-chain or branched alkenyl or alkynyl having in each case 3 to 6 carbon atoms, or represents in each case optionally cyano-, carboxyl-, halogen- or C₁-C₃-alkoxy-carbonyl-substituted cycloalkyl or cycloalkylalkyl having in each case 3 to 6 carbon atoms in the cycloalkyl group and optionally 1 to 3 carbon atoms in the alkyl moiety.

R³ preferably represents hydrogen, optionally cyano-, carboxyl-, halogen-, C₁-C₃-alkoxy- or C₁-C₃-alkoxy-carbonyl-substituted straight-chain or branched alkyl having 1 to 6 carbon atoms, in each case optionally cyano-, carboxyl-, halogen- or C₁-C₃-alkoxy-carbonyl-subtituted straight-chain or branched alkenyl or alkynyl having in each case 3 to 6 carbon atoms, in each case optionally cyano-, carboxyl-, halogen- or C₁-C₃-alkoxy-carbonyl-substituted cycloalkyl or cycloalkylalkyl having in each case 3 to 6 carbon atoms in the cycloalkyl group and optionally 1 to 3 carbon atoms in the alkyl moiety, or represents in each case optionally nitro-, cyano-, carboxyl-, carbamoyl-, C₁-C₄-alkyl-, C₁-C₃-halogenoalkyl-, C₁-C₃-alkoxy-, C₁-C₃-halogenoalkoxy- or C₁-C₃-alkoxy-carbonyl-substituted aryl or arylalkyl having in each case 6 or 10 carbon atoms in the aryl group and optionally 1 to 3 carbon atoms in the alkyl moiety.

R⁴ preferably represents hydrogen or represents optionally cyano-, fluorine-, chlorine-, methoxy-, ethoxy-, n or i-propoxy-substituted alkyl having 1 to 4 carbon atoms.

R⁵ preferably represents hydrogen or alkyl having 1 to 4 carbon atoms.

A¹ particularly preferably represents a single bond or represents methylene (—CH₂—), ethylidene (ethane-1,1-diyl, —CH(CH₃)—), propylidene (propane-1,1-diyl, —CH(C₂H₅)—) or propane-2,2-diyl (—C(CH₃)₂—).

A² particularly preferably represents a single bond or represents methylene (—CH₂—), ethylidene (ethane-1,1-diyl, —CH(CH₃)—), dimethylene (ethane-1,2-diyl, —CH₂CH₂—), propylidene (propane-1,1-diyl, —CH(C₂H₅)—), propane-1,2-diyl (—CH(CH₃)—CH₂—), propane-2,2-diyl (—C(CH₃)₂—), trimethylene (propane-1,3-diyl, —CH₂CH₂CH₂—) or represents in each case optionally cyano-, fluorine-, chlorine-, methoxy-, ethoxy-, n- or i-propoxy-, methoxycarbonyl-, ethoxycarbonyl- or n- or i-propoxycarbonyl-substituted ethene-1,2-diyl, propene-1,2-diyl, propene-1,3-diyl, 1-butene-1,4-diyl or 2-butene-1,4-diyl.

R particularly preferably represents hydrogen, cyano, fluorine, chlorine, bromine, or represents in each case optionally fluorine- and/or chlorine-substituted methyl or ethyl.

X particularly preferably represents hydrogen, fluorine or chlorine.

Y Particularly preferably represents O (oxygen) or S (sulphur).

Z particularly preferably represents cyano or one of the groupings —CQ¹-O—R¹, —CQ²-N(R²,R³), —C(R⁴)═O, —C(R⁴)═N—O—R⁵, or represents alkylsulphonyl, alkoxysulphonyl or dialkylaminosulphonyl having in each case 1 to 3 carbon atoms in the alkyl groups.

R¹ particularly preferably represents hydrogen, in each case optionally cyano-, carboxyl-, fluorine-, chlorine, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl, n- or i-propoxy-carbonyl-substituted propenyl, butenyl, pentenyl, propynyl, butynyl or pentynyl, in each case optionally cyano-, carboxyl-, fluorine-, chlorine-, methoxycarbonyl-, ethoxycarbonyl-, n- or i-propoxy-carbonyl-substituted cyclopropyl, cyclobutyl, cyclopentyl, cyclohexyl, cyclopropylmethyl, cyclobutylmethyl, cyclopentylmethyl or cyclohexylmethyl, or represents in each case optionally nitro-, cyano-, carboxyl-, carbamoyl-, methyl-, ethyl-, n- or i-propyl-, n-, i-, s- or t-butyl-, difluoromethyl-, trifluoromethyl-, dichloromethyl-, trichloromethyl-, methoxy-, ethoxy-, n- or i-propoxy-, difluoromethoxy-, trifluoromethoxy-, fluoroethoxy-, difluoroethoxy-, trifluoroethoxy-, chloroethoxy-, dichloroethoxy-, methoxycarbonyl-, ethoxycarbonyl-, n- or i-propoxy-carbonyl-substituted phenyl, naphthyl, phenylmethyl or phenylethyl.

R² particularly preferably represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, pentenyl, propynyl, butynyl or pentynyl, or represents in each case optionally cyano-, carboxyl-, fluorine-, chlorine-, methoxycarbonyl-, ethoxycarbonyl-, n- or i-propoxycarbonyl-substituted cyclopropyl, cyclobutyl, cyclopentyl, cyclohexyl, cyclopropylmethyl, cyclobutylmethyl, cyclopentylmethyl or cyclohexylmethyl.

R³ particularly preferably represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, pentenyl, propynyl, butynyl or pentynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclobutyl, cyclopentyl, cyclohexyl, cyclopropylmethyl, cyclobutylmethyl, cyclopentylmethyl or cyclohexylmethyl, or represents in each case optionally nitro-, cyano-, carboxyl-, carbamoyl, methyl-, ethyl-, n- or i-propyl-, n-, i-, s- or t-butyl-, difluoromethyl-, trifluoromethyl-, dichloromethyl-, trichloromethyl-, methoxy-, ethoxy-, n- or i-propoxy-, difluoromethoxy-, trifluoromethoxy-, fluoroethoxy-, difluoroethoxy-, trifluoroethoxy-, chloroethoxy-, dichloroethoxy-, methoxycarbonyl-, ethoxycarbonyl-, n- or i-propoxy-carbonyl-substituted phenyl, naphthyl, phenylmethyl or phenylethyl.

R⁴ particularly preferably represents hydrogen or represents in each case optionally cyano-, fluorine-, chlorine-, methoxy-, ethoxy-, n or i-propoxy-substituted methyl, ethyl, n- or i-propyl.

R particularly preferably represents hydrogen or represents methyl, ethyl, n- or i-propyl.

A¹ very particularly preferably represents a single bond or represents methylene (—CH₂—), ethylidene (ethane-1,1-diyl, —CH(CH₃)—), propylidene (propane-1,1-diyl, —CH(C₂H₅)—) or propane-2,2-diyl (—C(CH₃)₂—).

A² very particularly preferably represents a single bond or represents methylene (—CH₂—), ethylidene (ethane-1,1-diyl, —CH(CH₃)—), dimethylene (ethane-1,2-diyl, CH₂CH₂—), propylidene (propane-1,1-diyl, —CH(C₂H₅)—) or represents in each case optionally cyano-, fluorine-, chlorine-, methoxy- or ethoxy-substituted ethene-1,2-diyl, propene-1,2-diyl or propane-1,3-diyl.

R very particularly preferably represents hydrogen, chlorine, bromine or optionally fluorine- and/or chlorine-substituted methyl.

X very particularly preferably represents hydrogen or fluorine.

Y very particularly preferably represents O (oxygen).

Z very particularly preferably represents cyano or one of the groupings —CQ¹-O—R¹, —CQ²-N(R²,R³), —C(R⁴)═O, —C(R⁴)═N—O—R⁵, or represents methylsulphonyl, ethylsulphonyl, n- or i-propylsulphonyl, methoxysulphonyl, ethoxysulphonyl, n- or i-propoxysulphonyl, dimethylaminosulphonyl or diethylaminosulphonyl.

R¹ very particularly preferably represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl-, n- or i-propoxy-carbonyl-substituted propenyl, butenyl, propynyl or butynyl, in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl.

R² very particularly preferably represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl.

R³ very particularly preferably represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl.

R⁴ very particularly preferably represents hydrogen or represents in each case optionally fluorine-, chlorine-, methoxy-, ethoxy-, n- or i-propoxy-substituted methyl, ethyl, n- or i-propyl.

R⁵ very particularly preferably represents hydrogen or represents methyl or ethyl, n- or i-propyl.

A¹ more preferably represents a single bond or represents methylene (—CH₂).

A² more preferably represents a single bond or methylene (—CH₂) or in each case optionally chlorine-substituted propene-1,2-diyl or propene-1,3-diyl.

R more preferably represents hydrogen or optionally fluorine- or chlorine-substituted methyl.

Z more preferably represents cyano or one of the groupings —CQ¹—O—R¹ or —C(R⁴)═O, or represents methylsulphonyl or ethylsulphonyl.

R¹ more preferably represents hydrogen, in each case optionally cyano-, fluorine-, chlorine-, methoxy- or ethoxy-substituted methyl, ethyl, n- or i-propyl, n-, i-, s- or t-butyl or represents in each case optionally cyano-, fluorine- or chlorine-substituted propenyl, butenyl, propynyl or butynyl.

R² more preferably represents hydrogen or represents in each case optionally cyano-, fluorine- or chlorine-substituted methyl, ethyl or n- or i-propyl.

R³ more preferably represents hydrogen, in each case optionally cyano-, fluorine-, chlorine-, methoxy- or ethoxy-substituted methyl, ethyl, n- or i-propyl, n-, i-, s- or t-butyl or represents in each case optionally cyano-, fluorine-, chlorine-, bromine-, methoxycarbonyl- or ethoxycarbonyl-substituted propenyl, butenyl, propynyl or butynyl.

Preference according to the invention is given to the compounds of the formula (I) which contain a combination of the meanings listed above as being preferred.

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

Most preference according to the invention is given to the compounds of the formula (I) which contain a combination of the meanings listed above as being most preferred.

A very particularly preferred group are those compounds of the formula (I) in which

A¹ represents a single bond,

A² represents a single bond or represents methylene (—CH₂—), ethylidene (ethane-1,1-diyl, —CH(CH₃)—) or dimethylene (ethane-1,2-diyl, —CH₂CH₂—), Q represents O (oxygen) or S (sulphur),

R represents hydrogen, chlorine, bromine or optionally fluorine- and/or chlorine-substituted methyl,

X represents hydrogen, fluorine or chlorine,

Y represents O (oxygen) or S (sulphur), and

Z represents cyano or one of the groupings —CQ¹-O—R¹, —CQ²-N(R²,R³), —C(R⁴)═O, —C(R⁴)═N—O—R⁵, or represents methylsulphonyl, ethylsulphonyl, n-or i-propylsulphonyl, methoxysulphonyl, ethoxysulphonyl, n- or i-propoxy-sulphonyl, dimethylaminosulphonyl or diethylaminosulphonyl, where

R¹ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl-, n- or i-propoxy-carbonyl-substituted propenyl, butenyl, propynyl or butynyl, in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,

R² represents hydrogen, in each case optionally cyano-, carboxyl-, fluorine, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl,

R³ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxycarbonyl-substituted phenyl, phenylmethyl or phenylethyl,

R⁴ represents hydrogen or represents in each case optionally fluorine-, chlorine-, methoxy-, ethoxy-, n- or i-propoxy-substituted methyl, ethyl, n- or i-propyl, and

R⁵ represents hydrogen or represents methyl, ethyl, n- or i-propyl.

A further very particularly preferred group are those compounds of the formula (I) in which

A¹ represents methylene (—CH₂—), ethylidene (ethane-1,1-diyl, —CH(CH₃)—), propylidene (propane-1,1-diyl, —CH(C₂H₅)—) or propane-2,2-diyl (—C(CH₃)₂—),

A² represents a single bond or represents methylene (—CH₂—), ethylidene (ethane-1,1-diyl, —CH(CH₃)—) or dimethylene (ethane-1,2-diyl, —CH₂CH₂—),

Q represents O (oxygen),

R represents hydrogen, chlorine, bromine or optionally fluorine- and/or chlorine-substituted methyl,

X represents hydrogen, fluorine or chlorine,

Y represents O (oxygen), and

Z represents cyano or one of the groupings —CQ¹-O—R¹, —CQ²-N(R²,R³), —C(R⁴)═O, —C(R⁴)═N—O—R⁵, or represents methylsulphonyl, ethylsulphonyl, n- or i-propylsulphonyl, methoxysulphonyl, ethoxysulphonyl, n- or i-propoxysulphonyl, dimethylaminosulphonyl or diethylaminosulphonyl, where

R¹ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl-, n- or i-propoxy-carbonyl-substituted propenyl, butenyl, propynyl or butynyl, in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,

R² represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl,

R³ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,

R⁴ represents hydrogen or represents in each case optionally fluorine-, chlorine-, methoxy-, ethoxy-, n- or i-propoxy-substituted methyl, ethyl, n- or i-propyl, and

R⁵ represents hydrogen or represents methyl, ethyl, n- or i-propyl.

The general or preferred radical definitions given above apply both to the end products of the formula (I) and, correspondingly, to the starting materials or intermediates required in each case for the preparation. These radical definitions can be combined with one another as desired, i.e. including combinations between the given preferred ranges.

The novel substituted benzo-nitrogen heterocycles of the formula (I) have interesting biological properties; in particular, they have strong herbicidal activity.

The novel substituted benzo-nitrogen heterocycles of the formula (I) are obtained when

(a) aminated benzo-nitrogen heterocylces of the general formula (II)

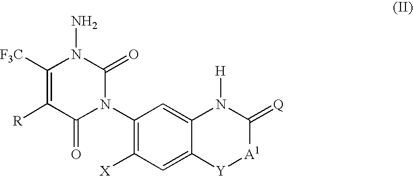

in which

A¹, Q, R, X and Y are as defined above

are reacted with electrophilic compounds of the general formula (III)

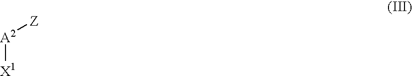

in which

A² and Z are as defined above and

X¹ represents halogen or the grouping —O-A²-Z,

if appropriate in the presence of a reaction auxiliary and if appropriate in the presence of a diluent,

or when

(b) benzo-nitrogen heterocycles of the general formula (IV)

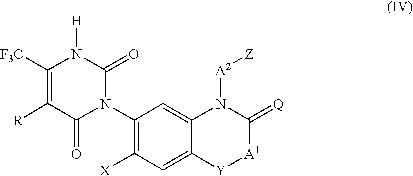

in which

A¹, A², Q, R, X, Y and Z are as defined above

are reacted with an electrophilic aminating agent,

if appropriate in the presence of a reaction auxiliary and if appropriate in the presence of a diluent.

Using, for example, 1-amino-3-(7-fluoro-3,4-dihydro-3-oxo-2H-1,4-benzoxazine-6-yl)-6-trifluoromethyl-2,4-(1H,3H)-pyrimidinedione and chloroacetonitrile as starting materials, the course of the reaction in the process (a) according to the invention can be illustrated by the formula scheme below:

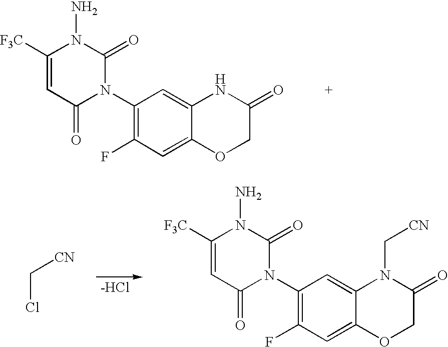

Using, for example, 6-(3,6-dihydro-2,6-dioxo-4-trifluoromethyl-1 (2H)-pyrimidynyl)-7-fluoro-2,3-dihydro-3-oxo-4H-1,4-benzoxazine-4-acetonitrile and 1-aminooxy-2,4-dinitrobenzene as starting materials, the course of the reaction in the process (b) according to the invention can be illustrated by the formula scheme below:

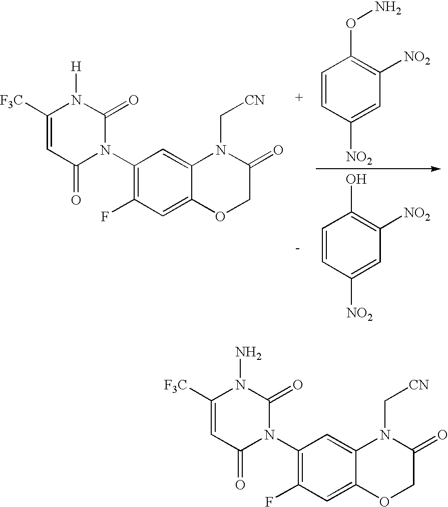

The formula (II) provides a general definition of the aminated benzo-nitrogen heterocycles to be used as starting materials in the process (a) according to the invention for preparing compounds of the general formula (I). In the general formula (II), A¹, Q, R, X and Y preferably or in particular have those meanings which have already been mentioned above, in connection with the description of the compounds of the general formula (I) according to the invention, as being preferred or as being particularly preferred for A¹, Q, R, X and Y.

Examples of starting materials of the formula (III) which may be mentioned with preference are: 1-amino-3-(3,4-dihydro-3-oxo-2H-1,4-benzoxazine-6-yl)-6-trifluoromethyl-2,4-(1H,3H)-pyrimidinedione, 1-amino-3-(7-fluoro-3,4-dihydro-3-oxo-2H-1,4-benzoxazine-6-yl)-6-trifluoromethyl-2,4-(1H,3H)-pyrimidinedione, 1-amino-3-(2,3-dihydro-2-oxo-1,3-benzothiazol-5-yl)-6-trifluoromethyl-2,4(1H,3H)-pyrimidinedione and 1-amino-3-(6-fluoro-2,3-dihydro-2-oxo-1,3-benzothiazol-5-yl)-6-trifluoromethyl-2,4(1H,3H)-pyrimidinedione.

The starting materials of the general formula (II) have hitherto not been disclosed in the literature; as novel substances, they also form part of the subject-matter of the present application.

The novel aminated benzo-nitrogen heterocycles of the general formula (II) are obtained when benzo-nitrogen heterocycles of the general formula (V)

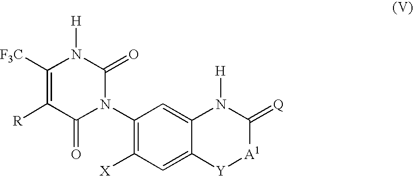

in which

A¹, Q, R, X and Y are as defined above

are reacted with an electrophilic aminating agent, such as, for example, 1-aminooxy-2,4-dinitro-benzene or 2-aminooxysulphonyl-1,3,5-trimethylbenzene, if appropriate in the presence of a reaction auxiliary, such as, for example, sodium bicarbonate or potassium carbonate, and if appropriate in the presence of a diluent, such as, for example tetrahydrofurane, ethyl acetate or N,N-dimethylformamide, at temperatures between −30° C. and +60° C. (cf. the preparation examples).

The precursors of the general formula (V) are known and/or can be prepared by processes known per se (cf. U.S. Pat. No. 5,127,935, cf. the preparation examples).

The formula (III) provides a general definition of the electrophilic compounds further to be used as starting materials in the process (a) according to the invention for preparing compounds of the general formula (I). In the general formula (III), A² and Z preferably have those meanings which have already been mentioned above, in connection with the description of the compounds of the general formula (I) according to the invention, as being preferred, particularly preferred, very particularly preferred or most preferred for A² and Z; X¹ preferably represents fluorine, chlorine, bromine, iodine or the grouping —O-A²-Z, in particular represents chlorine, bromine or iodine. The starting materials of the general formula (m) are known organic chemicals for synthesis.

The formula (IV) provides a general definition of the benzo-nitrogen heterocycles to be used as starting materials in the process (b) according to the invention for preparing compounds of the general formula (I). In the general formula (IV), A¹, A², Q, R, X, Y and Z preferably have those meanings which have already been mentioned above, in connection with the description of the compounds of the general formula (I) according to the invention, as being preferred, particularly preferred, very particularly preferred or most preferred for A¹, A², Q, R, X, Y and Z.

The starting materials of the general formula (IV) are known and/or can be prepared by processes known per se (cf. U.S. Pat. No. 5,084,084).

The process (b) according to the invention for preparing compounds of the general formula (I) is carried out using an electrophilic animating agent. Here, it is possible to use the customary electrophilic aminating agents, i.e. substances suitable for introducing an amino group. Examples which may be mentioned are 1-aminooxy-2,4-dinitrobenzene (2,4-dinitrophenyl-hydroxylamine), 2-aminooxysulphonyl-1,3,5-trimethylbenzene (O-mesitylenesulphonyl-hydroxylamine) and hydroxylamine-O-sulphonic acid.

The processes (a) and (b) according to the invention for preparing the compounds of the formula (I) are preferably carried out in the presence of a suitable reaction auxiliary. Here, suitable reaction auxiliaries are generally in each case the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide n- or i-propoxide, n-, i-, s- or t-butoxide; furthermore also basic organic nitrogen compounds, such as, for example, trimethylamine, triethylamine, tripropylamine, tributylamine, ethyl diisopropylamine, N,N-dimethylcyclohexylamine, dicyclohexylamine, ethyl dicyclohexylamine, N,N-dimethylaniline, N,N-dimethyl-benzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN), and 1,8-diazabicyclo[5.4.0]undec-7-ene (DBU).

The processes (a) and (b) according to the invention for preparing the compounds of the formula (I) are preferably carried out in the presence of a diluent. Here, suitable diluents are in each case generally the customary organic solvents. These preferably include aliphatic, alicyclic and aromatic, optionally halogenated hydrocarbons, such as, for example, pentane, hexane, heptane, petroleum ether, ligroine, benzine, benzene, toluene, xylene, chlorobenzene, dichlorobenzene, cyclohexane, methylcyclohexane, dichloromethane (methylenchloride), trichloromethane (chloroform) or carbon tetrachloride, dialkyl ethers, such as, for example, diethyl ether, diisopropyl ether, methyl t-butyl ether, ethyl t-butyl ether, methyl t-pentyl ether (MTBE), ethyl t-pentyl ether, tetrahydrofuran (THF), 1,4-dioxane, ethylene glycol dimethyl ether or ethylene glycol diethyl ether, diethylene glycol dimethyl ether or diethylene glycol diethyl ether; dialkyl ketones, such as, for example, acetone, butanone (methyl ethyl ketone), methyl i-propyl ketone or methyl i-butyl ketone, nitriles, such as, for example, acetonitrile, propionitrile, butyronitrile or benzonitrile; amides, such as, for example, N,N-dimethylformamide (DMF), N,N-dimethylacetamide, N-methylformanilide, N-methylpyrrolidone or Hexamethylphosphoric triamide; esters, such as, for example, methyl acetate, ethyl acetate, n- or i-propyl acetate, n-, i- or s-butyl acetate; sulphoxide, such as, for example, dimethyl sulphoxide; alkanols, such as, for example, methanol, ethanol, n- or i-propanol, n-, 1-, s- or t-butanol, ethylene glycol monomethyl ether or ethylene glycol monethyl ether, diethylene glycol monomethyl ether or diethylene glycol monoethyl ether; mixtures thereof with water or pure water.

When carrying out the processes (a) and (b) according to the invention, the reaction temperatures can in each case be varied within a relatively large range. In general, the processes are carried out at temperatures between −20° C. and +100° C., preferably between 0° C. and 80° C., in particular between 10° C. and 60° C.

The processes (a) and (b) according to the invention are generally carried out under atmospheric pressure. However, it is also possible to carry out the processes according to the invention under elevated or reduced pressure—in general between 0.1 bar and 10 bar.

To carry out the processes (a) and (b) according to the invention, the starting materials are generally employed in each case in approximately equimolar amounts. However, it is also possible to use a relatively large excess of one of the components in each case. The reactions are generally carried out in a suitable diluent in the presence of a reaction auxiliary, and the reaction mixture is generally stirred at the required temperature for a number of hours. Work-up is carried out by customary methods (cf. the preparation examples).

The active compounds according to the invention can be used as defoliants, desiccants, haulm killers and, especially, as weed killers. Weeds in the broadest sense are understood to mean all plants which grow in locations where they are undesired. Whether the substances according to the invention act as total or selective herbicides depends essentially on the amount used.

The active compounds according to the invention can be used, for example, in connection with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindemia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but also extends in the same manner to other plants.

The active compounds according to the invention are suitable, depending on the concentration, for the total control of weeds, for example on industrial terrain and rail tracks, and on paths and areas with and without tree plantings. Similarly, the active compounds according to the invention can be employed for controlling weeds in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds of the formula (I) according to the invention have strong herbicidal activity and a broad activity spectrum when used on the soil and on above-ground parts of plants. To a certain extent they are also suitable for the selective control of monocotyledonous and dicotyledonous weeds in monocotyledonous and dicotyledonous crops, both by the pre-emergence and by the post-emergence method.

At certain concentrations or application rates, the active compounds according to the invention can also be employed for controlling animal pests and fungal or bacterial plant diseases. If appropriate, they can also be used as intermediates or precursors for the synthesis of further active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, environment or storage space by the customary treatment-methods, for example by immersion, spraying, evaporation, fogging, scattering, painting on and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are: for example ammonium salts and ground natural minerals such as kaolins, clays, talc, chalk, quartz, attapulgite, montmorillonite or diatomaceous earth, and ground synthetic materials such as highly-dispersed silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignin-sulphite waste liquors and methylcellulose.

Tackifiers such as carboxymethylcellulose and natural and synthetic polymers in the form of powders, granules or latices, such as gum arabic, polyvynyl alcohol and polyvynyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic colorants such alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

For controlling weeds, the active compounds according to the invention, as such or in their formulations, can also be used as mixtures with known herbicides and/or substances which improve the compatibility with crop plants (“safeners”), finished formulations or tank mixes being possible. Also possible are mixtures with weed-killers comprising one or more known herbicides and a safener.

Possible components for the mixtures are known herbicides, for example

acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazon, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (—P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (—P-ethyl), fentrazamide, flamprop (-isopropyl, -isopropyl-L, -methyl), flazasulfuron, florasulam, fluazifop (—P-butyl), fluazolate, flucarbazone (-sodium), flufenacet, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, —P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, lactofen, lenacil, linuron, MCPA, mecoprop, mefenacet, mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, pentoxazone, phenmedipham, picolinafen, piperophos, pretilachlor, primisulfuron (-methyl), profluazol, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop (—P-ethyl, —P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

Further suitable for the mixtures are known safeners, for example AD-67, BAS-145138, benoxacor, cloquintocet (-mexyl), cyometrinyl, 2,4-D, DKA-24, dichlormid, dymron, fenclorim, fenchlorazol (-ethyl), flurazole, fluxofenim, furilazole, isoxadifen (-ethyl), MCPA, mecoprop (—P), mefenpyr (-diethyl), MG-191, oxabetrinil, PPG-1292, R-29148.

A mixture with other known active compounds, such as fungicides, insecticides, acaricides, nematicides, bird repellents, plant nutrients and agents which improve soil structure, is also possible.

The active compounds can be used as such, in the form of their formulations or in the use forms prepared therefrom by further dilution, such as ready-to-use solutions, suspensions, emulsions, powders, pastes and granules. They are used in a customary manner, for example by watering, spraying, atomizing or broadcasting.

The active compounds according to the invention can be applied both before and after emergence of the plants. They can also be incorporated into the soil before sowing.

The amount of active compound used can vary within a relatively wide range. It depends essentially on the nature of the desired effect. In general, the amounts used are between 1 g and 10 kg of active compound per hectare of soil surface, preferably between 5 g and 5 kg per ha.

As already mentioned above, it is possible to treat all plants and their parts according to the invention. In a preferred embodiment, wild plant species and cultivars, or those obtained by conventional biological breeding, such as crossing or protoplast fusion, and parts thereof, are treated. In a further preferred embodiment, transgenic plants and cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the cultivars which are in each case commercially available or in use are treated according to the invention. Cultivars are to be understood as meaning plants having certain properties (“traits”) and which have been obtained by conventional breeding, by mutagenesis or by recombinant DNA techniques. They can be varieties, bio- or genotypes.

Depending on the plant species or cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention—also in combination with other agrochemical active compounds, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fingi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinothricin (for example the “PAT” gene). The genes which impart the desired traits in question in each case can also be present in combinations with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosates, for example maize, cotton, soya bean), Liberty Link® (tolerance to phosphinothricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to cultivars having these genetic traits or genetic traits still to be developed, which cultivars will be developed and/or marketed in the future.

The plants listed can be treated according to the invention in a particularly advantageous manner with the compounds of the general formula I or the active compound mixtures according to the invention where, in addition to the effective control of the weed plants the abovementioned synergistic effects with the transgenic plants or plant cultivars occur. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

### Example 1

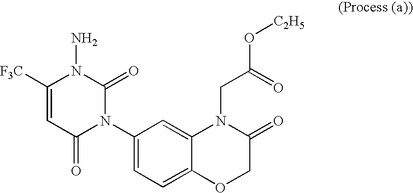

At room temperature (about 20° C.), 0.86 ml (3.3 mmol) of anhydrous potassium carbonate is added to a solution of 0.86 g (2.5 mmol) of 1-amino-3-(3,4-dihydro-3-oxo-2H-1,4-benzoxazin-6-yl)-6-trifluoromethyl-2,4(1H,3H)-pyrimidinedione in 14 ml acetonitrile. The suspension is stirred for 5 minutes, 0.42 g (2.5 mmol) of ethyl bromoacetate are added and the mixture is then heated at 70° C. for 4 hours. After the reaction has ended, the mixture is diluted with water and extracted with ethyl acetate. The organic phase is separated off, washed successively with water and saturated aqueous sodium chloride solution, dried over magnesium sulphate and freed from the solvent under reduced pressure. The resulting crude product is tritiated with a mixture of ether/petroleum ether (1:1), filtered and dried at 60° C. under reduced pressure.

This gives 0.95 g (89% of theory) of ethyl 6-(3-amino-3,6-dihydro-2,6-dioxo-4-trifluoromethyl-1 (2H)-pyrimidynyl)-2,3-dihydro-3-oxo-4H-1,4-benzoxazine-4-acetate.

logP (at pH=2): 1.90.

### Example 2

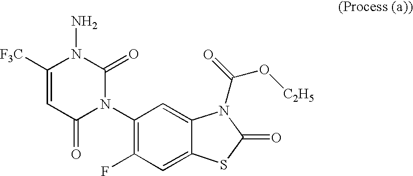

0.30 g (0.8 mmol) 1-amino-3-(6-fluoro-2,3-dihydro-2-oxo-1,3-benzothiazol-5-yl)-6-trifluoromethyl-2,4(1H,3H)-pyrimidinedione is dissolved in 20 ml of acetonitrile, 0.17 g (1.2 mmol) of potassium carbonate is added and the suspension is stirred at room temperature (about 20° C.) for 15 minutes. 0.13 g (1.2 mmol) of ethyl chloroformate is then added to the reaction mixture, which is stirred at room temperature overnight. The reaction mixture is then concentrated under reduced pressure, and 20 ml of water and 20 ml of diethyl ether are added to the residue. The organic phase is separated off, dried over sodium sulphate and concentrated under reduced pressure. The residue is stirred with 10 ml of petroleum ether and 1 ml of diethyl ether, and the resulting crystalline product is isolated by filtration with suction.

0.2 g (52% of theory) of ethyl 5-(3-amino-3,6-dihydro-2,6-dioxo-4-trifluoromethyl-1(2H)-pyrimidynyl)-6-fluoro-2-oxo-3(2H)-benzothiazole carboxylate.

logP (at pH=2): 2.44.

### Example 3

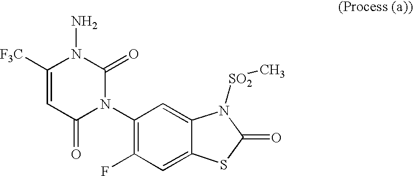

0.40 g (1.1 mmol) 1-amino-3-(6-fluoro-2,3-dihydro-2-oxo-1,3-benzothiazol-5-yl)-6-trifluoromethyl-2,4(1H,3H)-pyrimidinedione is dissolved in 20 ml of acetonitrile, and 0.17 g (1.5 mmol) of methanesulphonate chloride is added. At room temperature (about 20° C.), 0.15 g (1.5 mmol) of triethylamine—in 2 ml of acetonitrile—is then added dropwise. The solution is stirred at room temperature overnight. 25 ml of water are then added to the reaction mixture, which is extracted twice with 25 ml of ethyl acetate. The organic phase is dried over sodium sulphate and concentrated under reduced pressure. The residue is stirred with 10 ml of diethyl ether and the resulting crystalline product is isolated by filtration with suction.

This gives 0.22 g (40% of theory) 1-amino-3-(6-fluoro-2,3-dihydro-3-methylsulphonyl-2-oxo-5-benzothiazolyl)-6-trifluoromethyl-2,4(1H,3H)-pyrimidinedione.

logP (at pH=2): 2.16.

Analogously to Examples 1 to 3 and in accordance with the general description of the preparation processes according to the invention, it is also possible to prepare, for example, the compounds of the general formula (I) listed in Table 1 below.

TABLE 1

| | |
|---|---|
| | (I) |
| 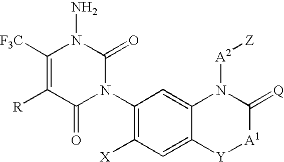 | |

Examples of compounds of the formula (I)

| Ex. | | | | | | | Physical |
|---|---|---|---|---|---|---|---|
| No. | A¹ | Q | R | X | Y | A²—Z | data |
| 4 | — | O | H | F | S | CH₂CN | logP = |
| | | | | | | | 1.97^{a)} |
| 5 | CH₂ | O | H | F | O | CH₂CN | logP = |
| | | | | | | | 1.83^{a)} |
| 6 | CH₂ | O | H | H | O | CH₂CN | logP = |
| | | | | | | | 1.57^{a)} |
| 7 | CH₂ | O | H | F | O | CH₂COOCH₃ | logP = |
| | | | | | | | 1.90^{a)} |
| 8 | CH₂ | O | H | F | O | CH₂COOC₂H₅ | logP = |
| | | | | | | | 2.14^{a)} |
| 9 | CH₂ | O | H | F | O | CH₂COOC₄H₉-t | logP = |
| | | | | | | | 2.60^{a)} |
| 10 | CH₂ | O | H | F | O | CH₂COOC₃H₇-i | logP = |
| | | | | | | | 2.38^{a)} |
| 11 | CH₂ | O | H | H | O | CH₂COOCH₃ | |
| 12 | CH₂ | O | H | H | O | CH₂COOC₃H₇-i | logP = |
| | | | | | | | 2.14^{a)} |
| 13 | CH₂ | O | H | H | O | CH₂COOC₄H₉-t | logP = |
| | | | | | | | 2.37^{a)} |
| 14 | — | O | H | F | S | COCH₃ | logP = |
| | | | | | | | 2.36^{a)} |
| 15 | — | O | H | F | S | SO₂C₂H₅ | |
| 16 | — | O | H | F | S | CH₂COOCH₃ | |
| 17 | — | O | H | F | S | CH₂COOC₃H₇-i | |
| 18 | — | O | H | F | S | CH₂COOC₂H₅ | |
| 19 | — | O | H | F | S | CH₂COOC₄H₉-t | |
| 20 | — | O | H | F | S | COOC₄H₉-t | |
| 21 | — | O | CF₃ | F | S | C(CH₂Cl)═CHCOOH | |
| 22 | — | O | H | F | S | C(CH₂Cl)═CHCOOCH₃ | logP = |
| | | | | | | | 2.46^{a)} |
| 23 | — | O | H | F | S | COOC₄H₉-i | logP = |
| | | | | | | | 3.17^{a)} |
| 4 | — | O | H | F | S | CH₂CN | logP = |
| | | | | | | | 1.97^{a)} |
| 24 | — | O | H | F | S | C(CF₃)═CHCOOC₂H₅ | logP = |
| | | | | | | | 2.98^{a)} |
| 25 | — | O | H | F | S | CH₂CH═CHCOOC₂H₅ | logP = |
| | | | | | | | 2.51^{a)} |
| 26 | — | O | H | F | S | C(CH₃)═CHCOOC₂H₅ | (E/Z |
| | | | | | | | mix- |
| | | | | | | | ture) |
| 27 | — | O | H | F | S | 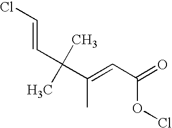 | logP =3.14^{a)}(E/Z mix- ture) |
| 28 | — | O | H | F | S | C(CH₃)═CHCOOC₂H₅ | (E |
| | | | | | | | isomer) |
| 29 | — | O | H | F | S | C(CH₃)═CHCOOC₂H₅ | (Z |
| | | | | | | | isomer) |
| 30 | — | O | H | F | S | CN | logP = |
| | | | | | | | 2.19^{a)} |
| 31 | CH₂ | O | H | H | O | C(CH₃)═CHCOOC₂H₅ | (E/Z |
| | | | | | | | mix- |
| | | | | | | | ture) |

LogP values given in Table 1 were determined in accordance with EC Directive 79/831 Annex V.A8 by HPLC (High Performance Liquid Chromatography) using a reversed-phase column (C 18). Temperature: 43° C.

(a) Mobile phases for the determination in the acidic range: 0.1% aqueous phosphoric acid, acetonitrile; linear gradient from 10% acetonitrile to 90% acetonitrile—the corresponding measurement results in Table 1 are marked ^{a)}.

(b) Mobile phases for the determination in the neutral range: 0.01 molar aqueous phosphate buffer solution, acetonitrile; linear gradient from 10% acetonitrile to 90% acetonitrile—the corresponding measurement results in Table 1 are marked ^{b)}.

Calibration was carried out using unbranched alkan-2-ones (having 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

Starting Materials of the Formula (II):

### Example (II-1)

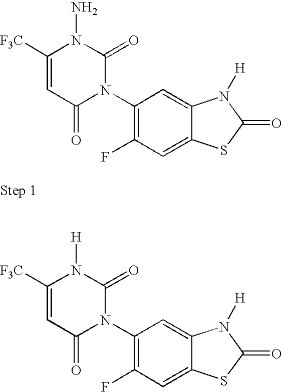

13 g (70.6 mmol) of 5-amino-6-fluoro-2-oxo-3(2H)-benzothiazole are dissolved in 400 ml of ethyl acetate and heated at 70° C. 15.5 g (78.1 mmol) of trichloromethyl chloroformate are then added dropwise and the mixture is heated at reflux for 3 hours and concentred under reduced pressure. This gives 14.8 g (70 mmol) of 6-fluoro-2-oxo-3(2H)-benzothiazole 5-isocyanate as residue.

Under argon, 5.6 g (140 mmol) of sodium hydride (60%) are suspended in 150 ml of N,N-dimethylformamide, 14.2 g (77 mmol) of ethyl 3-amino-4,4,4-trifluorocrotonate are added and the mixture is stirred at from 20° C. to 30° C. for 1 hour. The reaction solution is cooled to −60° C., 14.8 g (70 mmol) of 6-fluoro-2-oxo-3(2H)-benzothiazole 5-isocyanate—suspended in 200 ml of toluene—are added dropwise, and the mixture is then stirred at room temperature for 4 hours. The reaction mixture is poured into 200 ml of water and extracted three times with in each case 150 ml of ethyl acetate and the aqueous phase is acidified using about 50 ml of dilute hydrochloric acid and extracted three times with in each case 150 ml of ethyl acetate. The combined organic phases are washed three times with in each case 150 ml of water, dried over sodium sulphate and concentrated under reduced pressure. The residue is taken up in 200 ml diethyl ether and admixed with such an amount of ethyl acetate that the residue is just dissolved. The product is then precipitated using petroleum ether, filtered off with suction, washed three times with in each case 150 ml of petroleum ether and dried.

This gives 18.2 g (68% of theory) of 3-(6-fluoro-2,3-dihydro-2-oxo-1,3-benzothiazol-5-yl)-6-trifluoromethyl-2,4(1H,3H)-pyrimidinedione

logP (at pH=2): 1.48.

Step 2

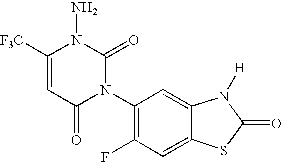

5.0 g (14.4 mmol) of 3-(6-fluoro-2,3-dihydro-2-oxo-1,3-benzothiazol-5-yl)-6-trifluoromethyl-2,4(1H,3H)-pyrimidinedione are dissolved in 50 ml of N,N-dimethylformamide, 1.3 g (16 mmol) of sodium bicarbonate are added and the mixture is stirred at room temperature for 30 minutes. 1.8 g (8.4 mmol) of 1-aminooxy-2,4-dinitrobenzene are then added, and the mixture is stirred at room temperature overnight. Another 1.8 g (8.4 mmol) of 1-aminooxy-2,4-dinitrobenzene are then added. The reaction mixture is stirred for another 2 days, 0.5 g (6 mmol) of sodium bicarbonate and 1.2 g (5.6 mmol) of 1-aminooxy-2,4-dinitrobenzene are added, and the mixture is stirred for another 4 days. The reaction mixture is mixed with 150 ml of water and extracted three times with in each case 150 ml of ether acetate, and the combined organic phases are washed twice with in each case 150 ml of water. The organic phase is dried over sodium sulphate and concentrated under reduced pressure. The residue is purified by column chromatography (silica gel, cyclohexane/ethyl acetate, 2:1 v/v).

This gives 3.1 g (58% of theory) of 1-amino-3-(6-fluoro-2,3-dihydro-2-oxo-1,3-benzothiazol-5-yl)-6-trifluoromethyl-2,4(1H,3H)-pyrimidinedione.

logP (at pH=2): 1.66.

Analogously to Example (II-1), it is also possible, for example, to prepare the compounds of the general formula (II) listed in Table 2 below.

TABLE 2

| | |
|---|---|
| | (II) |
| 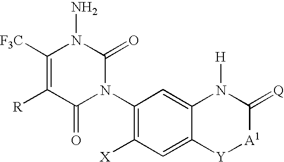 | |

Examples of compounds of the formula (II)

| Ex. | | | | | | Physical |
|---|---|---|---|---|---|---|
| No. | A¹ | Q | R | X | Y | Data |
| II-2 | CH₂ | O | H | H | O | logP = 1.22^{a)} |
| II-3 | CH₂ | O | H | F | O | logP = 1.57^{a)} |
| II-4 | CH₂ | O | Cl | F | O | |
| II-5 | CH₂ | O | CH₃ | F | O | |
| II-6 | CH₂ | O | Cl | H | O | |
| II-7 | CH₂ | O | CH₃ | H | O | |
| II-8 | — | O | H | H | S | |
| II-9 | — | O | Cl | F | S | |
| II-10 | — | O | CH₃ | F | S | |
| II-11 | — | O | Cl | H | S | |
| II-12 | — | O | CH₃ | H | S | |

Use examples:

### Example A

Pre-Emergence Test

| | |
|---|---|
| Solvent: | 5 parts by weight of acetone |
| Emulsifier: | 1 part by weight of alkylaryl polyglycol ether |

To produce a suitable preparation of active compound, one part by weight of active compound is mixed with the stated amount of solvent, the stated amount of emulsifier is added and the concentrate is diluted with water to the desired concentration.

Seeds of the test plants are sown in normal soil. After 24 hours, the soil is sprayed with the preparation of active compounds such that the particular amount of active compound desired is applied per unit area. The concentration of active compound in the spray liquor is chosen such that the particular amount of active compound desired is applied in 1000 litres of water per hectare.

After three weeks, the degree of damage to the plants is rated in % damage in comparison to the development of untreated control. The figures denote:

0%=no effect (like untreated control)

100%=total destruction

In this test, for example, the compounds of Preparation Examples 1, 5, 6, 7, 8, 9, 10, 11 and 12 show very strong action against weeds.

### Example B

Post-Emergence Test

| | |
|---|---|
| Solvent: | 5 parts by weight of acetone |
| Emulsifier: | 1 part by weight of alkylaryl polyglycol ether |

To produce a suitable preparation of active compound, one part by weight of active compound is mixed with the stated amount of solvent, the stated amount of emulsifier is added and the concentrate is diluted with water to the desired concentration.

Test plants of a height of 5-15 cm are sprayed with the preparation of active compounds such that the particular amounts of active compounds desired are applied per unit area. The concentration of the spray liquor is chosen such that the particular amounts of active compounds desired are applied in 1000 l of water/ha.

After three weeks, the degree of damage to the plants is rated in % damage in comparison to the development of the untreated control.

The figures denote:

0%=no effect (like untreated control)

100%=total destruction

In this test, for example, the compounds of Preparation Examples 1, 5, 6, 7, 8, 9, 10, 11 and 12 show very strong action against weeds.

1. Compounds of the formula (I) 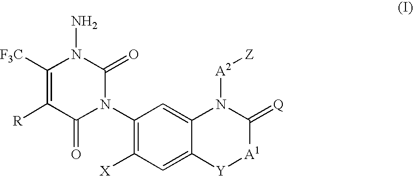
in which
A¹ represents a single bond or represents straight-chain or branched alkanediyl having 1 to 4 carbon atoms,
A² represents a single bond or represents straight-chain or branched alkanediyl having 1 to 4 carbon atoms or represents optionally cyano-, carboxyl-, halogen-, C₁-C₄-alkoxy or C₁-C₄-alkoxy-carbonyl-substituted alkenediyl having 2 to 6 carbon atoms,
Q represents O (oxygen) or S (sulphur),
R represents hydrogen, cyano, halogen or optionally halogen-substituted straight-chain or branched alkyl having 1 to 4 carbon atoms,
X represents hydrogen or halogen,
Y represents O (oxygen), S (sulphur), NH or N-(C₁-C₄-alkyl), and
Z represents cyano, one of the groupings —CQ¹-O—R¹, —CQ²-N(R²,R³), —C(R⁴)═O, —C(R⁴)═N—O—R⁵, or represents alkylsulphonyl, alkoxysuphonyl or dialkylaminosulphonyl having in each case 1 to 6 carbon atoms in the alkyl groups, where
Q¹ represents O (oxygen) or S (sulphur),
Q² represents O (oxygen) or S (sulphur),
R¹ represents hydrogen, optionally cyano-, carboxyl-, halogen-, C₁-C₄ alkoxy- or C₁-C₄-alkoxy-carbonyl-substituted straight-chain or branched alkyl having 1 to 10 carbon atoms, in each case optionally cyano-, carboxyl-, halogen- or C₁-C₄-alkoxycarbonyl-substituted straight-chain or branched alkenyl or alkynyl having in each case 3 to 10 carbon atoms, in each case optionally cyano-, carboxyl-, halogen- or C₁-C₄-alkoxycarbonyl-substituted cycloalkyl or cycloalkylalkyl having in each case 3 to 6 carbon atoms in the cycloalkyl group and optionally 1 to 4 carbon atoms in the alkyl moiety, or represents in each case optionally nitro-, cyano-, carboxyl-, carbamoyl-, C₁-C₄-alkyl-, C₁-C₄-halogenoalkyl, C₁-C₄-alkoxy, C₁-C₄-halogenoalkoxy- or C₁-C₄-alkoxy-carbonyl-substituted aryl or arylalkyl having in each case 6 or 10 carbon atoms in the aryl group and optionally 1 to 4 carbon atoms in the alkyl moiety,
R² represents hydrogen, optionally cyano-, carboxyl-, halogen-, C₁-C₄-alkoxy- or C₁-C₄-alkoxy-carbonyl-substituted straight-chain or branched alkyl having 1 to 10 carbon atoms, in each case optionally cyano-, carboxyl-, halogen- or C₁-C₄-alkoxycarbonyl-substituted straight-chain or branched alkenyl or alkynyl having in each case 3 to 10 carbon atoms, or represents in each case optionally cyano-, carboxyl-, halogen- or C₁-C₄-alkoxy-carbonyl-substituted cycloalkyl or cycloalkylalkyl having in each case 3 to 6 carbon atoms in the cycloalkyl group and optionally 1 to 4 carbon atoms in the alkyl moiety,
R³ represents hydrogen, optionally cyano-, carboxyl, halogen-, C₁-C₄-alkoxy- or C₁-C₄-alkoxy-carbonyl-substituted straight-chain or branched alkyl having 1 to 10 carbon atoms, in each case optionally cyano-, carboxyl-, halogen- or C₁-C₄-alkoxycarbonyl-substituted straight-chain or branched alkenyl or alkynyl having in each case 3 to 10 carbon atoms, in each case optionally cyano-, carboxyl-, halogen- or C₁-C₄-alkoxycarbonyl-substituted cycloalkyl or cycloalkylalkyl having in each case 3 to 6 carbon atoms in the cycloalkyl group and optionally 1 to 4 carbon atoms in the alkyl moiety, or represents in each case optionally nitro-, cyano-, carboxyl-, carbamoyl-, C₁-C₄-alkyl-, C₁-C₄-halogenoalkyl-, C₁-C₄-alkoxy-, C₁-C₄-halogenoalkoxy- or C₁-C₄-alkoxy-carbonyl-substituted aryl or arylalkyl having in each case 6 or 10 carbon atoms in the aryl group and optionally 1 to 4 carbon atoms in the alkyl moiety,
R⁴ represents hydrogen or represents optionally cyano-, halogen- or C₁-C₄-alkoxy-substituted alkyl having 1 to 6 carbon atoms, and
R⁵ represents hydrogen or alkyl having 1 to 6 carbon atoms.

2. Compounds of the formula (I) according to claim 1, characterized in that
A¹ represents a single bond or represents straight-chain or branched alkanediyl having 1 to 3 carbon atoms,
A² represents a single bond or represents straight-chain or branched alkanediyl having 1 to 3 carbon atoms or represents optionally cyano-, carboxyl-, fluorine-, chlorine-, bromine-, methoxy-, ethoxy-, n- or i-propoxy-, n-, i-, s- or t-butoxy-, methoxycarbonyl-, ethoxycarbonyl-, n- or i-propoxycarbonyl-, n-, i-, s- or t-butoxycarbonyl-substituted alkenediyl having 2 to 4 carbon atoms,
Q represents O (oxygen),
R represents hydrogen, cyano, halogen or optionally halogen-substituted straight-chain or branched alkyl having 1 to 3 carbon atoms,
X represents hydrogen, fluorine, chlorine or bromine,
Y represents O (oxygen), S (sulphur) or NH,
Z represents cyano, one of the groupings —CQ¹-O—R¹, —CQ²-N(R²,R³), —C(R⁴)═O, —C(R⁴)═N—O—R⁵, or represents alkylsulphonyl, alkoxysulphonyl or dialkylaminosulphonyl having in each case 1 to 4 carbon atoms in the alkyl groups,
Q¹ represents O (oxygen),
Q² represents O (oxygen),
R¹ represents hydrogen, optionally cyano-, carboxyl-, halogen-, C₁-C₃-alkoxy- or C₁-C₃-alkoxy-carbonyl-substituted straight-chain or branched alkyl having 1 to 6 carbon atoms, in each case optionally cyano-, carboxyl-, halogen- or C₁-C₃-alkoxy-carbonyl-substituted straight-chain or branched alkenyl or alkynyl having in each case 3 to 6 carbon atoms, in each case optionally cyano-, carboxyl-, halogen- or C₁-C₃-alkoxy-carbonyl-substituted cycloalkyl or cycloalkylalkyl having in each case 3 to 6 carbon atoms in the cycloalkyl group and optionally 1 to 3 carbon atoms in the alkyl moiety, or represents in each case optionally nitro-, cyano-, carboxyl-, carbamoyl-, C₁-C₄-alkyl-, C₁-C₃-halogenoalkyl-, C₁-C₃-alkoxy-, C₁-C₃-halogenoalkoxy- or C₁-C₃-alkoxy-carbonyl-substituted aryl or arylalkyl having in each case 6 or 10 carbon atoms in the aryl group and optionally 1 to 3 carbon atoms in the alkyl moiety,
R² represents hydrogen, optionally cyano-, carboxyl-, halogen-, C₁-C₃-alkoxy- or C₁-C₃-alkoxy-carbonyl-substituted straight-chain or branched alkyl having 1 to 6 carbon atoms, in each case optionally cyano-, carboxyl-, halogen- or C₁-C₃-alkoxy-carbonyl-substituted straight-chain or branched alkenyl or alkynyl having in each case 3 to 6 carbon atoms, or represents in each case optionally cyano-, carboxyl-, halogen- or C₁-C₃-alkoxy-carbonyl-substituted cycloalkyl or cycloalkylalkyl having in each case 3 to 6 carbon atoms in the cycloalkyl group and optionally 1 to 3 carbon atoms in the alkyl moiety,
R³ represents hydrogen, optionally cyano-, carboxyl-, halogen-, C₁-C₃-alkoxy- or C₁-C₃-alkoxy-carbonyl-substituted straight-chain or branched alkyl having 1 to 6 carbon atoms, in each case optionally cyano-, carboxyl-, halogen- or C₁-C₃-alkoxy-carbonyl-subtituted straight-chain or branched alkenyl or alkynyl having in each case 3 to 6 carbon atoms, in each case optionally cyano-, carboxyl-, halogen- or C₁-C₃-alkoxy-carbonyl-substituted cycloalkyl or cycloalkylalkyl having in each case 3 to 6 carbon atoms in the cycloalkyl group and optionally 1 to 3 carbon atoms in the alkyl moiety, or represents in each case optionally nitro-, cyano-, carboxyl-, carbamoyl-, C₁-C₄-alkyl-, C₁-C₃-halogenoalkyl-, C₁-C₃-alkoxy-, C₁-C₃-halogenoalkoxy- or C₁-C₃-alkoxy-carbonyl-substituted aryl or arylalkyl having in each case 6 or 10 carbon atoms in the aryl group and optionally 1 to 3 carbon atoms in the alkyl moiety,
R⁴ represents hydrogen or represents optionally cyano-, fluorine-, chlorine-, methoxy-, ethoxy-, n- or i-propoxy-substituted alkyl having 1 to 4 carbon atoms, and
R⁵ represents hydrogen or alkyl having 1 to 4 carbon atoms.

3. Compounds of the formula (I) according to claim 1 or 2, characterized in that
A¹ represents a single bond or represents methylene (—CH₂—), ethylidene (ethane-1,1-diyl, —CH(CH₃)—), propylidene (propane-1,1-diyl, —CH(C₂H₅)—) or propane-2,2-diyl (—C(CH₃)₂—),
A² represents a single bond or represents methylene (—CH₂—), ethylidene (ethane-1,1-diyl, —CH(CH₃)—), dimethylene (ethane-1,2-diyl, —CH₂CH₂—), propylidene (propane-1,1-diyl, —CH(C₂H₅)—), propane-1,2-diyl (—CH(CH₃)—CH₂—), propane-2,2-diyl (—C(CH₃)₂—), trimethylene (propane-1,3-diyl, —CH₂CH₂CH₂—) or represents in each case optionally cyano-, fluorine-, chlorine-, methoxy-, ethoxy-, n- or i-propoxy-, methoxycarbonyl-, ethoxycarbonyl- or n- or i-propoxycarbonyl-substituted ethene-1,2-diyl, propene-1,2-diyl, propene-1,3-diyl, 1-butene-1,4-diyl or 2-butene-1,4-diyl,
R represents hydrogen, cyano, fluorine, chlorine, bromine, or represents in each case optionally fluorine- and/or chlorine-substituted methyl or ethyl,
X represents hydrogen, fluorine or chlorine,
Y represents O (oxygen) or S (sulphur),
Z represents cyano or one of the groupings —CQ¹-O—R, —CQ²-N(R²,R³), —C(R⁴)═O, —C(R⁴)═N—O—R⁵, or represents alkylsulphonyl, alkoxysulphonyl or dialkylaminosulphonyl having in each case 1 to 3 carbon atoms in the alkyl groups,
R¹ represents hydrogen, in each case optionally cyano-, carboxyl-, fluorine-, chlorine, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl, n- or i-propoxy-carbonyl-substituted propenyl, butenyl, pentenyl, propynyl, butynyl or pentynyl, in each case optionally cyano-, carboxyl-, fluorine-, chlorine-, methoxycarbonyl-, ethoxycarbonyl-, n- or i-propoxy-carbonyl-substituted cyclopropyl, cyclobutyl, cyclopentyl, cyclohexyl, cyclopropylmethyl, cyclobutylmethyl, cyclopentylmethyl or cyclohexylmethyl, or represents in each case optionally nitro-, cyano-, carboxyl-, carbamoyl-, methyl-, ethyl-, n- or i-propyl-, n-, i-, s- or t-butyl-, difluoromethyl-, trifluoromethyl-, dichloromethyl-, trichloromethyl-, methoxy-, ethoxy-, n- or i-propoxy-, difluoromethoxy-, trifluoromethoxy-, fluoroethoxy-, difluoroethoxy-, trifluoroethoxy-, chloroethoxy-, dichloroethoxy-, methoxycarbonyl-, ethoxycarbonyl-, n- or i-propoxy-carbonyl-substituted phenyl, naphthyl, phenylmethyl or phenylethyl,
R² represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, pentenyl, propynyl, butynyl or pentynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclobutyl, cyclopentyl, cyclohexyl, cyclopropylmethyl, cyclobutylmethyl, cyclopentylmethyl or cyclohexylmethyl,
R³ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, pentenyl, propynyl, butynyl or pentynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclobutyl, cyclopentyl, cyclohexyl, cyclopropylmethyl, cyclobutylmethyl, cyclopentylmethyl or cyclohexylmethyl, or represents in each case optionally nitro-, cyano-, carboxyl-, carbamoyl-, methyl-, ethyl-, n- or i-propyl-, n-, i-, s- or t-butyl-, difluoromethyl-, trifluoromethyl-, dichloromethyl-, trichloromethyl-, methoxy-, ethoxy-, n- or i-propoxy-, difluoromethoxy-, trifluoromethoxy-, fluoroethoxy-, difluoroethoxy-, trifluoroethoxy-, chloroethoxy-, dichloroethoxy-, methoxycarbonyl-, ethoxycarbonyl-, n- or i-propoxy-carbonyl-substituted phenyl, naphthyl, phenylmethyl or phenylethyl,
R⁴ represents hydrogen or represents in each case optionally cyano-, fluorine-, chlorine-, methoxy-, ethoxy-, n or i-propoxy-substituted methyl, ethyl, n- or i-propyl, and
R⁵ represents hydrogen or represents methyl, ethyl, n- or i-propyl.

4. Compounds of the formula (I) according to any of claims 1 to 4, characterized in that
A¹ represents a single bond or represents methylene (—CH₂—), ethylidene (ethane-1,1-diyl, —CH(CH₃)—), propylidene (propane-1,1-diyl, —CH(C₂H₅)—) or propane-2,2-diyl (—C(CH₃)₂—),
A² represents a single bond or represents methylene (—CH₂—), ethylidene (ethane-1,1-diyl, —CH(CH₃)—), dimethylene (ethane-1,2-diyl, CH₂CH₂—), propylidene (propane-1,1-diyl, —CH(C₂H₅)—) or represents in each case optionally cyano-, fluorine-, chlorine-, methoxy- or ethoxy-substituted ethene-1,2-diyl, propene-1,2-diyl or propane-1,3-diyl,
R represents hydrogen, chlorine, bromine or optionally fluorine- and/or chlorine-substituted methyl,
X represents hydrogen or fluorine,
Y represents O (oxygen),
Z represents cyano or one of the groupings —CQ¹-O—R¹, —CQ²-N(R²,R³), —C(R⁴)═O, —C(R⁴)═N—O—R⁵, or represents methylsulphonyl, ethylsulphonyl, n- or i-propylsulphonyl, methoxysulphonyl, ethoxysulphonyl, n- or i-propoxysulphonyl, dimethylaminosulphonyl or diethylaminosulphonyl,
R¹ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl-, n- or i-propoxy-carbonyl-substituted propenyl, butenyl, propynyl or butynyl, in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,
R² represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl,
R³ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxycarbonyl-substituted phenyl, phenylmethyl or phenylethyl,
R⁴ represents hydrogen or represents in each case optionally fluorine-, chlorine-, methoxy-, ethoxy-, n- or i-propoxy-substituted methyl, ethyl, n- or i-propyl, and
R⁵ represents hydrogen or represents methyl or ethyl, n- or i-propyl.

5. Compounds of the formula (I) according to any of claims 1 to 4, characterized in that
A¹ represents a single bond or represents methylene (—CH₂),
A² represents a single bond or methylene (—CH₂) or in each case optionally chlorine-substituted propene-1,2-diyl or propene-1,3-diyl,
R represents hydrogen or optionally fluorine- or chlorine-substituted methyl,
Z represents cyano or one of the groupings —CQ¹—O—R¹ or —C(R⁴)═O, or represents methylsulphonyl or ethylsulphonyl,
R¹ represents hydrogen, in each case optionally cyano-, fluorine-, chlorine-, methoxy- or ethoxy-substituted methyl, ethyl, n- or i-propyl, n-, i-, s- or t-butyl or represents in each case optionally cyano-, fluorine- or chlorine-substituted propenyl, butenyl, propynyl or butynyl,
R² represents hydrogen or represents in each case optionally cyano-, fluorine- or chlorine-substituted methyl, ethyl or n- or i-propyl, and
R³ represents hydrogen, in each case optionally cyano-, fluorine-, chlorine-, methoxy- or ethoxy-substituted methyl, ethyl, n- or i-propyl, n-, i-, s- or t-butyl or represents in each case optionally cyano-, fluorine-, chlorine-, bromine-, methoxycarbonyl- or ethoxycarbonyl-substituted propenyl, butenyl, propynyl or butynyl.

6. Compounds of the formula (I) according to any of claims 1 to 5, characterized in that
A¹ represents a single bond,
A² represents a single bond or represents methylene (—CH₂—), ethylidene (ethane-1,1-diyl, —CH(CH₃)—) or dimethylene (ethane-1,2-diyl, —CH₂CH₂—),
Q represents O (oxygen) or S (sulphur),
R represents hydrogen, chlorine, bromine or optionally fluorine- and/or chlorine-substituted methyl,
X represents hydrogen, fluorine or chlorine,
Y represents O (oxygen) or S (sulphur), and
Z represents cyano or one of the groupings —CQ¹-O—R¹, —CQ²-N(R²,R³), —C(R⁴)═O, —C(R⁴)═N—O—R⁵, or represents methylsulphonyl, ethylsulphonyl, n- or i-propylsulphonyl, methoxysulphonyl, ethoxysulphonyl, n- or i-propoxysulphonyl, dimethylaminosulphonyl or diethylaminosulphonyl, where
R¹ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl-, n- or i-propoxy-carbonyl-substituted propenyl, butenyl, propynyl or butynyl, in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,
R² represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl,
R³ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,
R⁴ represents hydrogen or represents in each case optionally fluorine-, chlorine-, methoxy-, ethoxy-, n- or i-propoxy-substituted methyl, ethyl, n- or i-propyl, and
R⁵ represents hydrogen or represents methyl, ethyl, n- or i-propyl.

7. Compounds of the formula (I) according to any of claims 1 to 6, characterized in that
A¹ represents methylene (—CH₂—), ethylidene (ethane-1,1-diyl, —CH(CH₃)—), propylidene (propane-1,1-diyl, —CH(C₂H₅)—) or propane-2,2-diyl (—C(CH₃)₂—),
A² represents a single bond or represents methylene (—CH₂—), ethylidene (ethane-1,1-diyl, —CH(CH₃)—) or dimethylene (ethane-1,2-diyl, —CH₂CH₂—),
Q represents O (oxygen),
R represents hydrogen, chlorine, bromine or optionally fluorine- and/or chlorine-substituted methyl,
X represents hydrogen, fluorine or chlorine,
Y represents O (oxygen), and
Z represents cyano or one of the groupings —CQ¹-O—R¹, —CQ²-N(R²,R³), —C(R⁴)═O, —C(R⁴)═N—O—R⁵, or represents methylsulphonyl, ethylsulphonyl, n- or i-propylsulphonyl, methoxysulphonyl, ethoxysulphonyl, n- or i-propoxysulphonyl, dimethylaminosulphonyl or diethylaminosulphonyl, where
R¹ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, methoxycarbonyl-, ethoxycarbonyl-, n- or i-propoxy-carbonyl-substituted propenyl, butenyl, propynyl or butynyl, in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,
R² represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, or represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl,
R³ represents hydrogen, in each case optionally cyano-, carboxyl-, fluorine-, chlorine-, methoxy-, ethoxy-, n- or i-propoxy-, methoxycarbonyl-, ethoxycarbonyl-, n- or i-propoxy-carbonyl-substituted methyl, ethyl, n- or i-propyl, n-, i-, s- or t-butyl, in each case optionally cyano-, carboxyl-, fluorine-, chlorine-, bromine-, methoxycarbonyl-, ethoxycarbonyl-, n- or i-propoxy-carbonyl-substituted propenyl, butenyl, propynyl or butynyl, represents in each case optionally cyano-, carboxyl-, fluorine-, chlorine-, methoxycarbonyl-, ethoxycarbonyl-, n- or i-propoxy-carbonyl-substituted cyclopropyl, cyclopentyl, cyclohexyl, cyclopropylmethyl, cyclopentylmethyl or cyclohexylmethyl, or represents in each case optionally nitro-, cyano-, carboxyl-, carbamoyl-, methyl-, ethyl-, n- or i-propyl-, trifluoromethyl-, methoxy-, ethoxy-, n- or i-propoxy-, difluoromethoxy-, trifluoromethoxy-, methoxycarbonyl-, ethoxycarbonyl-, n- or i-propoxy-carbonyl-substituted phenyl, phenylmethyl or phenylethyl,
R⁴ represents hydrogen or represents in each case optionally fluorine-, chlorine-, methoxy-, ethoxy-, n- or i-propoxy-substituted methyl, ethyl, n- or i-propyl, and
R⁵ represents hydrogen or represents methyl, ethyl, n- or i-propyl.

8. Process for preparing compounds of the formula (I) according to any of claims 1 to 7, characterized in that
(a) aminated benzo-nitrogen heterocylces of the general formula (II) 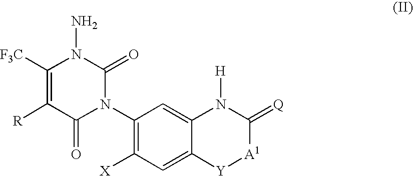
in which
A¹, Q, R, X and Y are as defined in any of claims 1 to 7
are reacted with electrophilic compounds of the general formula (III) 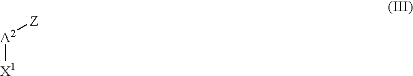
in which
A² and Z are as defined in any of claims 1 to 7 and
X¹ represents halogen or the grouping —O-A²-Z,
if appropriate in the presence of a reaction auxiliary and if appropriate in the presence of a diluent,
or in that
(b) benzo-nitrogen heterocycles of the general formula (I) 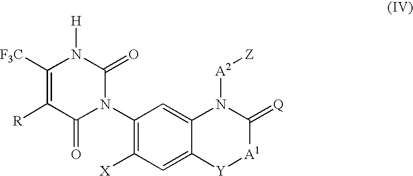
in which
A¹, A², Q, R, X, Y and Z are as defined in any of claims 1 to 7
are reacted with an electrophilic aminating agent,
if appropriate in the presence of a reaction auxiliary and if appropriate in the presence of a diluent.

9. Compounds of the formula (II) 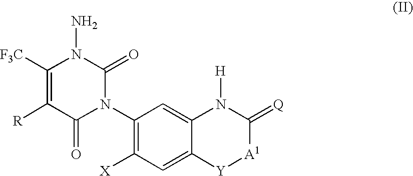
in which
A¹, Q, R, X and Y are as defined in any of claims 1 to 7.

10. Process for preparing compounds of the formula (II) according to claim 9, characterized in that benzo-nitrogen heterocycles of the general formula (V) 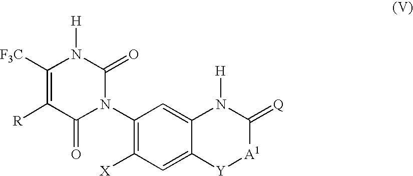
in which
A¹, Q, R, X and Y are as defined in any of claims 1 to 7
are reacted with an electrophilic aminating agent, if appropriate in the presence of a reaction auxiliary and if appropriate in the presence of a diluent, at temperatures between −30° C. and +60° C.

11. Herbicidal compositions, characterized in that they comprise at least one compound of the formula (I) according to any of claims 1 to 7 and customary extenders.

12. Use of one or more compounds of formula (I) according to any of claims 1 to 7 for controlling undesirable plants.

13. Process for controlling undesirable plants, characterized in that at least one compound of the formula (I) according to any of claims 1 to 7 is allowed to act on the undesirable plants and/or their habitat.

14. Use of compositions according to claim 11 for controlling undesirable plants.
